(19) United States
(12) Patent Application Publication
(10) Pub. No.: US 2020/0037649 A1
(43) Pub. Date: Feb. 6, 2020

(54) FOOD PRODUCT OR BEVERAGE CONTAINING A MARINE MICROALGA

(71) Applicant: ODONTELLA, BORDEAUX (FR)

(72) Inventors: Pierre CALLEJA, BORDEAUX (FR); Alain GUILLOU, BORDEAUX (FR)

(21) Appl. No.: 16/339,811

(22) PCT Filed: Oct. 5, 2017
(86) PCT No.: PCT/EP2017/075377

§ 371 Date: Apr. 5, 2019

(30) Foreign Application Priority Data
Oct. 5, 2016 (FR) 16 59604

(51) Int. Cl. A23L 33/105; A23L 27/00; A61K 35/02; A61K 36/03

(52) U.S. Cl. CPC A23L 33/105; A61K 36/03; A61K 35/02; A23L 27/00

(57) ABSTRACT

The subject of the invention is a product for human or animal consumption, in the form of food or drink, comprising between 0.2 and 50% by weight of biomass of a marine microalga and/or at least one extract of said microalga. The invention also relates to the use of this marine microalga and/or at least one extract of said microalga as a flavor booster and/or enhancer in food products or beverages.

### CROSS REFERENCE TO RELATED APPLICATIONS

This application is a U.S. national stage application of International Application No. PCT/EP2017/075377 filed Oct. 5, 2017, which claims priority to French patent application No. 1659604 filed Oct. 5, 2016. The contents of the applications are incorporated herein by reference.

### TECHNICAL FIELD

The present invention relates to food products and beverages comprising a marine microalga. The invention also relates to the use of this marine microalga as a booster and/or flavor enhancer in foods or beverages.

### BACKGROUND

Microalgae are freshwater or marine aquatic organisms with a high concentration of excellent quality nutrients, including whole proteins, polyunsaturated fatty acids, fiber and polysaccharides, minerals, water-soluble vitamins, pigments and antioxidants. In addition, they are first-tier organisms that accumulate relatively few contaminants and harmful compounds. Moreover, the production of microalgae uses cultures, which does not require harvesting in the natural environment unlike other aquatic organisms, and thus allows for better control of the quality and traceability of finished products.

For these reasons, some of these organisms have been suggested for some years as food supplements, in particular two species of a freshwater microalga of the genus Chlorella. The use of a cyanobacterium called spirulina has also been suggested.

However, these microorganisms do not contain certain compounds that are essential for health, particularly long-chain polyunsaturated fatty acids from the omega-3 group that are currently provided by products of animal origin (fish oil, krill, shrimp and molluscs especially) not accessible to a growing portion of the population (those with allergies, vegetarians, vegans, conscientious ecologists, etc.). In addition, these microorganisms do not contribute any flavor to food products or beverages.

### SUMMARY

The subject of this invention is to offer products intended as food based on a raw material of algal origin, the use of which makes it possible to simultaneously increase the levels of EPA and DHA, fucoxanthin and other antioxidant pigments in food products without the use of animal products, provide organic silica and enhance and/or improve the taste of these products by limiting the amount of sodium they contain. To this end, this invention relates to the use of a marine microalga and extract of a microalga of the Odontella genus in food products.

The marine microalgae of the genus Odontella are well known. Their use, in particular in cosmetics, has previously been described, for example in FR2918564, FR2833171.

Advantageously, their use in relation to this invention allows the content of EPA and DHA in food products to be increased, as well as the content of other compounds such as fucoxanthin, carotenoids with high oxidizing power and organic silica which is integrated into the bone matrix and allows for harder and more resistant bones. Surprisingly, the use of Odontella in food products enhances the taste and provides new flavors that enhance the marine flavors and tastes and increase the organoleptic quality of the product.

In particular, this invention relates to a product intended for human and animal consumption, in the form of food or beverages comprising between 0.2 and 50% by biomass weight of a marine microalga of the genus Odontella and/or at least one extract of said microalga.

This invention also relates to the use of the biomass of a marine microalga of the genus Odontella and/or at least one extract of said microalga as a flavor booster and/or enhancer in food products or beverages.

The invention will now be described in detail.

### DETAILED DESCRIPTION

### Definitions

“Biomass” of a marine microalga of the genus Odontella within the meaning of the invention means the entirety of the cellular material.

“Extract” within the meaning of the invention means any mixture of several native compounds contained in the Odontella biomass, obtained by one or more extraction processes.

Booster and/or flavor enhancer” effect within the meaning of the invention typically means marine aromas and flavors and/or a more pronounced marine aroma that increases the organoleptic qualities of the product.

### Detailed Description of the Invention

The invention relates to a product intended for human or animal consumption, in the form of food or drink, comprising between 0.2 and 50% by weight of a marine microalga of the genus Odontella.

The product intended for consumption described in the invention may be in particular:
a veterinary food or “pet food” intended for animal consumption, for example a specific feed for particular growth phases of the animal (nursery/reproduction stage, prevention of disease, growth etc.)
a veterinary drink intended for animal consumption, for example a dietary supplement specific to particular growth stages of the animal (nursery/reproduction stage, prevention of disease, growth etc.),
a food intended for human consumption, such as a cereal product, a bakery product, a sweet or salty cake, a sweet or savory biscuit, a candy, a soup, a vegetable crisp, a condiment, an oil, a cooked dish, a product processed from marine products (terrine, fish paste, etc.), a dairy product or a liquid derived from plants, whether fermented or not,
a drink for human consumption, such as a soda, a fruit juice, an energy drink, flavored water, a syrup or an alcoholic beverage.

The product described in the invention contains between 0.2 and 50% by weight:
of biomass of a marine microalga of the genus Odontella, and/or
at least one extract of a marine microalga of the genus Odontella.

The marine microalga(e) present in the products described in the invention may be of any species belonging to the genus Odontella, such as Odontella aurita, Odontella granulata, Odontella longicruris, Odontella mobiliensis, Odontella regia, Odontella rhombus, Odontella simensis.

Preferably, Odontella aurita is used.

Odontella biomass may be in unprocessed form, that is to say unprocessed microlagae recovered after production.

The production of Odontella may be carried out according to any suitable process.

In particular, the production of Odontella is carried out in an open environment (especially in “raceways”) exposed to the sun (photoautotrophy) but also in closed environments in photobioreactors or in plastic bags whose walls are transparent (photoautotrophy). Some strains may also be produced in fermentor bioreactors by heterotrophy/mixotrophy.

The raw biomass may be treated prior to its use in the food products described in the invention or before it is used to obtain extracts which are themselves then used in products intended as food.

The pretreatment of the biomass preferably includes a wash, which acts in particular to reduce its salt, and preferably sodium, content.

It may make use of a particular method which consists of the following steps:
Centrifugation of Odontella biomass, in particular with a vertical centrifuge with two-phase trays and/or a horizontal centrifuge,
Suspending with stirring for 5 to 30 minutes,
Separation of the solid and liquid phases preferably by centrifugation (two-phase centrifuge) and/or by filtration, in particular with tangential filtration (microfiltration and/or ultra-filtration and/or nanofiltration),
Recovery of the solid phase and drying preferably by lyophilization and/or zeodration.

Aerosol drying and/or spray drying may also be used.

The biomass, whether processed or not, is incorporated into the product intended as food or is used to obtain an extract.

The extract(s) contained in the products described in the invention may be of any type and be obtained by any suitable method.

According to one suitable production process, the product described in the invention comprises a lipid extract of Odontella. This extract can be used alone or with at least one or more other Odontella extracts (for example a hydrophilic extract) and/or Odontella biomass and/or one or more other extracts of other microalgae or cyanobacteria in the food product or drink.

The lipid extract preferably contains:
neutral lipids, in particular glycerides, mono-, di- and triglycerides, free fatty acids and sterols,
polar lipids, in particular glycolipids and phospholipids, and/or
lipophilic pigments, in particular carotenoids and chlorophylls.

Even more preferably, the lipid extract specifically comprises EPA, DHA, fucoxanthin, carotenoid pigments, water-soluble polysaccharides and/or phospholipids.

The lipid extract is preferably in liquid form but may be in solid form, particularly in the form of an encapsulated powder.

The lipid extract can be obtained by supercritical CO2 extraction with or without the use of a cosolvent (ethanol or ethyl acetate), and/or extraction with re-circulated hot ethanol, from biomass preferably pretreated by drying and/or washing and drying as previously described. This extraction may be followed by a microencapsulation stage to allow the use of lipid extracts in an aqueous medium. It is possible to envisage a complementary fractioning phase of the lipid extract for separation of phospholipids, neutral lipids (including fatty acids of nutritional interest), carotenoid pigments and/or sterols.

According to one variant, the products intended for feeding described in the invention may contain a hydrophilic extract.

This extract may be used alone or with at least one or more other extracts of Odontella (for example a lipid extract) and/or Odontella biomass and/or one or more other extracts of other microalgae or cyanobacteria in the food product or drink.

The hydrophilic extract preferably contains:
proteins,
free amino acids
mineral salts, in particular organic silica and trace minerals,
hydrophilic pigments, in particular polyphenols,
polysaccharides,
and/or fibers.

Even more preferably, the hydrophilic extract specifically contains organic silica, polyphenols and proteins.

The hydrophilic extract may be in solid form, especially in powder form, or in liquid form.

The hydrophilic extract may be obtained by extraction preferably with supercritical CO₂ with or without co-solvent with low-toxicity or GRAS solvents such as ethanol or ethyl acetate, from a biomass preferentially previously treated with drying and/or washing and drying as previously described. It is possible to envisage a complementary phase of fractioning of the extract.

The lipid and hydrophilic compounds can also be separated and recovered by a treatment in the liquid phase during the washing phase or following this desalination phase:
Stirring of the microalgae cells with stirring for 5 to 30 minutes, allowing a physical treatment to be used (microwaves and/or ultrasounds and/or thermal shocks and/or osmotic shocks) and/or enzymatic treatment (proteolytic enzymes and/or or lipolytic enzymes and/or enzymes responsible for the lysing of membrane polysaccharides).
Separation of solid phases and liquid phases (aqueous/lipid) preferably by centrifugation (three-phase centrifuge) and/or by filtration, in particular with tangential filtration (microfiltration and/or ultrafiltration and/or nanofiltration).
Recovery of the solid phase and drying preferably by lyophilization and/or zeodratation. Aerosol drying and/or spray drying may also be used.
Recovery of the lipid liquid phase (oil and fat) which can be used without any special treatment other than the addition of antioxidant compounds (vitamins E, aromatic plant extracts, etc.): raw or refined oil according to the techniques of the art (purification, discoloration, deodorization, addition of antioxidant compounds (vitamin E, aromatic plant extracts, etc.): refined or partially refined oil.

Preferentially, all the hydrophilic extracts comprise organic silica.

The hydrophilic extracts are particularly suitable for liquid products, in particular for drinks.

The products intended for consumption described in the invention are preferably obtained by mixing Odontella biomass in powder form and/or an Odontella extract in powder form or in liquid form. In the case where it is an encapsulated lipid extract, it can be introduced into liquid and solid products. For the protein extracts of Odontella, it is necessary to introduce them as a stable emulsion in liquid foods and as a dry powder in a solid food.

The presence of microalgae of the genus Odontella in foods or drinks described in the invention makes it possible on the one hand to provide the consumer with important nutrients for the maintenance of optimal health that compensate for the shortcomings of modern food, and on the other hand to bring a particular flavor to the foods or drinks in which they are added, including a taste or aroma of the ocean and/or the marine environment which is slightly sweet, fresh and low in iodine.

The invention therefore also relates to the use of biomass of a marine microalga of the genus Odontella and/or at least one extract of said microalga as a booster and/or a flavor enhancer in food and beverages.

The products described in the invention may, in addition to marine microalgae of the genus Odontella, contain biomass or extracts of microalgae of the genus Chlorella and/or biomass or extracts of Spirulina. Advantageously, such a mixture makes it possible to rebalance the protein content, to incorporate phycocyanins and to increase trace elements, minerals and vitamins, in particular in Group B, which is not very plentiful in Odontella.

The invention is now illustrated by a set of non-exhaustive examples.

### EXAMPLES

### Example 1: Sparkling Beverage

This example is a sparkling beverage with a mild, slightly sweet, fresh and slightly iodized taste or aroma of the ocean and/or the marine environment. The drink contains extracts of Odontella:
an encapsulated lipidic extract (powder) providing polyunsaturated fatty acids
a hydrophilic extract containing marine trace elements, organic silica, marine proteins including all essential acids and polysaccharides.

The drink also includes Spirulina and Chlorella.

The composition is presented in the table below:

| Ingredients | Amount |
|---|---|
| Sparkling water | 490 g |
| Hydrophilic extract of microencapsulated | 5 to 10 g |
| marine microalgae of the genus Odontella | |
| Organic Natural Strawberry Extracts | 2 to 4 g |
| Or Organic Natural Raspberry Extracts | 2 to 4 g |
| Or Organic Natural Cherry Extracts | 2 to 4 g |
| Or Organic Natural Hibiscus Extracts | 2 to 4 g |
| Naturex type concentrated rosemary | 0.5 to 1 g |
| extract (powerful antioxidant) | |
| Hydrophilic extract of Spirulina and/or | 0.5 to 2 g |
| Chlorella | |
| Organic cane sugar | 2 to 5 g |

### Example 2: Lactofermented Yogurt and Vegetable Preparations

This example is a lactofermented product containing a dry hydrophilic extract of Odontella totalling between 1 and 5% of the fresh weight of the product. This extract contains marine trace elements, organic silica, marine proteins and polysaccharides, pigments, antioxidants. The lactofermented product may also contain extracts of Spirulina and Chlorella.

### Example 3: Salad Dressing

This example is a condiment product of the salad dressing or table oil variety containing:
a stable emulsion of lipid extract of Odontella and Chlorella comprising polyunsaturated fatty acids and phospholipids,
a hydrophilic dry extract of Odontella containing marine oligoelements and organic silica.

### Example 4: Appetizer Biscuit

This example is an aperitif biscuit containing dry biomass of Odontella, Chlorella and Spirulina combined with extracts of Odontella rich in organic silica and proteins of marine origin.

1. A product intended for human or animal consumption, in the form of food or drink, comprising between 0.2 and 50% by weight of biomass of a marine microalga of the genus Odontella and/or at least one extract of said microalga.

2. The product according to claim 1, characterized as a cereal product, a bakery product, a sweet or savory cake, a sweet or savory biscuit, a candy, a soup, a vegetable crisp, a condiment, an oil, a cooked dish, a product processed from marine products, a dairy product.

3. The product according to claim 1, characterized as a soda, a fruit juice, an energy drink, a flavored water, a syrup or an alcoholic beverage.

4. The product of claim 1 comprising a lipid extract of Odontella.

5. The product of claim 1 comprising a lipid extract which contains neutral lipids, polar lipids and/or lipophilic pigments.

6. The product of claim 1 comprising a lipid extract comprising EPA, DHA, fucoxanthin, carotenoid pigments and/or phospholipids.

7. The product of claim 1 comprising a hydrophilic extract of Odontella.

8. The product of claim 7 wherein the hydrophilic extract contains proteins, mineral salts, hydrophilic pigments, polysaccharides and/or fibers.

9. The product of claim 7, wherein the hydrophilic extract comprising comprises organic silica, polyphenols and proteins.

10. The product of claim 1 comprising marine microalga Odontella aurita.

11. The product of claim 1 comprising the Odontella biomass having been previously treated.

12. The product of claim 1 comprising an extract of Odontella obtained from an Odontella biomass which has undergone prior treatment.

13. The product of claim 11, wherein the Odontella biomass is treated by a method consisting of the following steps:
centrifuging the Odontella biomass,
suspending the centrifuged Odontella biomass with stirring for 5 to 30 minutes,
separating the solid and liquid phases by centrifugation and/or filtration, and
recovering the solid and drying phase.

14. The product of claim 1, characterized by a particular flavor due to the presence of Odontella.

15. (canceled)

16. A method for flavoring or enhancing a flavor in a food product or beverage, the method comprising: flavoring the food product or beverage with a biomass of a marine microalga of the genus Odontella and/or at least one extract of said microalga.
